(19) United States
(12) Patent Application Publication
(10) Pub. No.: US 2022/0226250 A1
(43) Pub. Date: Jul. 21, 2022

(54) Additive Method For 3D Printing Active Ingredient-Containing Objects

(71) Applicant: DiHeSys Digital Health Systems GmbH, Schwäbisch-Gmünd (DE)

(72) Inventors: Markus Dachtler, Ulm (DE); Alexander Richter, Pocking (DE); Gerald Huber, Plüderhausen (DE)

(21) Appl. No.: 17/615,431

(22) PCT Filed: May 29, 2020
(86) PCT No.: PCT/EP2020/065096

§ 371 Date: Mar. 23, 2022

(30) Foreign Application Priority Data
May 31, 2019 (EP) 19177764.8

(51) Int. Cl. A61K 9/20

(52) U.S. Cl. CPC A61K 9/2095; B33Y 80/00

(57) ABSTRACT

The present invention relates to an additive method for the production of solid or semi-solid two- or three-dimensional objects containing one of more pharmaceutical active agent(s), as well as to the objects produced by said method, such as semi-solid or solid dosage forms or medical devices. The method is a three-dimensional printing method in which individual defined volume increments are printed, which can be selected essentially freely as regards contained active agents, used carrier materials, shape, size, color, concentrations of active agents, and arrangement in the produced object.

### FIELD OF THE INVENTION

The present invention relates to an additive method for the production of solid or semi-solid three-dimensional objects which contain one of more pharmaceutical active agent(s), as well as to the objects produced by said method, such as semi-solid or solid dosage forms or medical devices. The method is an additive, preferably three-dimensional, printing method in which individual defined volume increments are printed, which can be selected essentially freely as regards contained active agents, used carrier materials, shape, size, color, concentrations of active agents, and arrangement in the produced object.

### BACKGROUND OF THE INVENTION

An additive 3D printing process for producing pharmaceutical dosage forms by filament fusion fabrication (FFF) is known from WO 2016/038356 A1.

### SUMMARY OF THE INVENTION

The technical problem underlying the present invention is the provision of a method for producing active agent-containing objects such as pharmaceutical dosage forms or active agent-containing medical devices, which method enables higher flexibility in selecting different base materials, active agents and their distribution in the active agent-containing object.

The above technical problem is solved by the embodiments of the present invention as disclosed in the claims, the present description and the accompanying figures.

In particular, the present invention provides a method for the production of an object containing at least one pharmaceutical active agent, the method comprising:
(i) generating a two- or three-dimensional representation of the object to be produced using predefined volume increments;
(ii) printing a predefined volume increment on a building platform or on an object provided on the building platform;
(iii) printing a further volume increment in a manner such that volume increments at least partially contact or overlap each other; and
(iv) repeating steps (ii) and (iii) until the object is generated;
wherein at least one of the volume increments contains at least one pharmaceutical active agent and the volume increments comprise a base composition or base substance being flowable at a printing temperature acceptable for the at least one pharmaceutical agent, which base composition or base substance solidifies after printing of the respective volume increment and/or the volume increments are superficially glued together.

According to the invention, the term “three-dimensional object” is to be understood that in the real world any object produced by two- or three-dimensional processes, here especially pharmaceutical dosage forms and medical devices, extends in three spatial directions. In case during the method according to the invention only one layer of at least partially contacting volume increments is printed on the building platform or on an object already provided on the building platform, the method according to the invention can also be described as a 2D printing method. In case volume increments are applied e.g. as drops which, through the subsequent removal of moisture such as by drying, represent two-dimensional objects on a macroscopic scale, these are, however, on a microscopic scale three-dimensional structures such that, according to the invention, in such embodiments the object extends in three dimensions.

According to a preferred embodiment of the method according to the invention, the production of the object is carried out in layers i.e., the volume increments are printed in layers in steps (ii) and (iii). Preferably, the volume increments are printed line-by-line and file-by-file, respectively.

The present invention is particularly characterized by high flexibility in the composition and the enablement of a multitude of possible designs of the produced semi-solid or solid object. For example, in preferred embodiments of the method, different volume increments can comprise different active agents and/or different amounts of active agents and/or different base compositions or base substances, respectively. Furthermore, the shape and/or the volume of the volume increments (also denoted hereinafter as “voxels”) can be the same or different.

According to the invention, however, it is also contemplated that the object is made of volume increments all containing a single, identical active agent wherein it is also envisaged according to the invention that each volume increment can contain the same amount of the active agent or the same concentration of the active agent, respectively.

The volume increments can be chosen essentially freely and can be in the form of e.g., drops, spheres, points, cylinders, cubes, cuboids or other shapes wherein the mentioned geometric shapes (spheres, cylinders, cubes, cuboids) are understood according to the invention so as to mean that the voxels adopt this shape, preferably upon solidification after printing, such that preferred forms of voxels according to the invention which can be also more generally mentioned are pellets being preferably approximated to a spherical shape or to a cylinder, and granules. Preferred forms of voxels are therefore particularly drop-, pellet-, cylindrical and granule-shaped voxels. As already mentioned above the shape (such as the aforementioned examples) and the size of the volume of the volume increments can be combined essentially independently from each other in a free fashion.

Certain preferred embodiments of the method of the invention make use of the principally free choice of volumes of the printed volume increments. In principle, the volume of a pharmaceutical dosage forms decreases during its degradation in the direction to or at, respectively, the location of release. This causes the amount of released active agent per unit time to decrease. For attaining an in this respect as uniform as possible release of active agent during the course of degradation, it is envisaged according to the invention that the volume increments are printed in such a manner that the volume of the volume increments increases from the outside to the inside. According to the invention, this is realized by printing corresponding layers of volume increments wherein the volume of the volume increments increases from layer to layer or from group of layers of equal volume to further layers of equal volume from the outside to the inside.

As already outlined, with the aid of the method of the invention, different pharmaceutical active agents (so-called APIs, active pharmaceutical ingredients) can be contained in the object. Furthermore, several (i.e., two or more) active agents can be contained in the volume increments. It is also possible, of course, to print volume increments in such a manner that each volume increment contains an API, but different APIs (two or more) are present in separate volume increments. It is also possible to print volume increments containing different concentrations (i.e., amount of active agent per volume increment) of a pharmaceutical active agent. In this respect, the method can in one embodiment be designed in that active agent-containing volume increments are configured and printed in a manner such that at least a first group of contacting or overlapping active agent-containing volume increments contain the same amount of active agent, and at least a second group of contacting volume increments contain an amount of active agent being different from the amount of active agent of the first group. In this way, concentration gradients can be formed in an object produced by the method. Also, this embodiment of the invention is used in preferred variants of the invention for providing a uniform release of active agent, as has been described above for the increase of the volumes of the volume increments from the outside to the inside. For providing of a uniform as possible release of active agent or active agents, the volume increments are preferably printed in such a manner that the concentration of active agent, preferably in the volume increments, increases from the outside to the inside.

In producing objects according to the method of the invention groups of volume increments having different active agents can be formed. In this embodiment at least one group of active agent-containing volume increments can be present which contains a first pharmaceutical active agent, and at least a second group of active agent-containing volume increments can be present which contains a second pharmaceutical active agent being different from the first active agent. The different groups of volume increments can be printed in such a manner that they are combined within the object. That means that the volume increments of the first and/or the second group (as well as of each optional further groups, if more than two active agents shall be present in the object to be printed) are printed in such a manner that the volume increments of the respect group contact each other.

In a further embodiment of the invention, it is also envisaged that active agent-containing volume increments are printed in such a manner that they form one or more groups in the object being at least partially, alternatively completely, surrounded by active agent-free volume increments which separate or shield, respectively, the active agent-containing volume increments from the external environment such that e.g., an object is formed having an active agent-containing core, or at least an inner group of connected active agent-containing volume increments (or several inner groups of neighboring volume increments having the same or different active agents or having the same or different concentrations of active agents), around which active agent-free volume increments are arranged. The “external environment” can be the environment surrounding the object. The “external environment” around a core region or an inner group of volume increments being directly connected with each other, can presently also mean another region within the printed object i.e., in the object of the invention, active agent-free volume increments can surround at least partially, optionally completely, active agent-containing groups of volume increments or single or groups of volume increments containing e.g., a different (or several different) active agent(s), so as to form separation layers or separation regions between the differently equipped volume increments.

In preferred embodiments, such arrangements can be used for the spatial isolation of the individual active agent-containing volume increments e.g., for avoiding chemical instabilities of the individual active agents and/or to separate different active agents which are not compatible with each other (for example, because they react with each other or otherwise impair their structure and/effectiveness).

In other embodiments of the type mentioned above, drug abuse deterrent tablets or capsules can be provided, which avoid that e.g., an active agent (such as opioids or active agents having addiction potential) can obtained e.g., upon disintegration or in a different way, from a dosage form and then be misused. Thus, in preferred embodiments of the invention, groups or layers of volume increments containing the active agent(s) (such as the substances potentially suited for misuse mentioned above) are printed which are surrounded by groups or layers of volume increments containing a substance which abolishes the effect of the active agent(s), degrades the active agent(s) or at least limits, more preferably prevents, in other ways the potential misuse of the active agent(s). One or more groups or one or more layers of volume increments not containing an active agent and not containing a substance preventing misuse (in preferred embodiments these volume increments will merely contain the used base substance) can be provided between the groups (or layers) of the active agent(s) and substances preventing misuse and separating the active agent-containing volume increments from the substance(s) preventing misuse.

Furthermore, such embodiments using active agent-containing volume increments surrounded at least in part by active agent-free volume increments can be used according to the invention for example, for producing embodiments releasing the active agent(s) slowly such as retard tablets or capsules or enteric coated tablets or capsules.

Therefore, the method according to the invention can be used for producing objects, particularly pharmaceutical dosage forms, releasing the API(s) at a selected location or a selected region of application (so-called “drug targeting”) i.e., preferably for regulating the release of the drug(s) from the printed dosage from. Such embodiments thus serve for delivering the drug or drugs to the optimal effective location or targeted location such as (preferably) after oral administration. In certain embodiments of the invention it is thus provided that the volume increments are printed in a manner such that a core region of volume increments in a pharmaceutical dosage form contains one or more desired active agent(s), and one or more layers of volume increments are arranged around this core region (or around this core volume), which layer(s) are e.g., pH dependently degraded or dissolved in the intestine such that the core region is exposed to the external environment at the selected region of the intestine and there releases the active agent(s) by the pH dependent degradation of the outer layer(s). This is typically attained by known polymers (such as shellac, copolymers of methacrylic acid and methacryl methacrylate, modified celluloses etc.) contained in the building substance, having a pH dependent degradation or pH dependent solubility, respectively, which can be very finely tuned such that a pH dependent exposure of the active agent-containing core region can be provided according to the invention for any section of the intestine, particularly the small intestine (duodenum, jejunum and ileum). The provision of a targeted release at a defined effective location or target location such as the intestine or a selected section of the intestine is not limited to pH dependently degradable and pH dependently soluble, respectively, layers of volume increments having respective pH dependent degradable and pH dependent soluble, respectively, polymers contained in such building substances. Other alternative or additional mechanisms can be realized. Thus, the volume increments can be printed according to the invention in a manner such that one or more layers of volume increments are formed e.g., directly on an active agent-containing core region or on one or more optionally present intermediate layers, which volume increments have a building substance containing or consisting of a bacterially degradable portion. Bacterially degradable polymers known to the skilled person such as starches or celluloses are suitable for this purpose. Such layers are preferably used for release of active agents in the colon. In preferred embodiments, the layers mentioned above e.g., pH dependently degraded layers (one or more thereof) and bacterially degradable layers, can be combined. For example, dosage forms of combinations of pharmaceutical active agents can be printed wherein volume increments having a first active agent are arranged in a core region which is surrounded by one or more layers of volume increments having a building substance which contains (or consists of) bacterially degradable substances. Thereupon follow(s) one or more layers of volume increments containing a building substance which contains (or consists of) one or more pH dependently degradable polymers. Alternatively, it is possible in such an embodiment containing drug targeting layers that only the core region contains one or more active agent(s).

Since the method can also be carried out under sterile conditions, the printing method of the invention can also applied to the provision of implants and/or active agent releasing injections or active agent depots.

Furthermore, it also adds to the increased flexibility of the method according to the invention that very different materials (active agents and base compositions and substances, respectively) can be used for volume increments.

The binding between the individual applied volume increments can occur in different ways. For example, in an embodiment using a meltable material the binding between such voxels can occur by solidification after application onto the carrier structure wherein this can be carried out by various mechanisms such as simple cooling and/or chemically by known substances. Alternatively, the voxel material e.g., a suitable binder can be added to a dispersion or a solution, which binder provides curing of the voxel after application wherein the curing can occur through the binder e.g., by heat supplied by a heat source in the printing device such as light source, preferably a laser device. The curing by the binder can also occur chemically by corresponding starter molecules and/or light of a suitable wavelength, the latter being preferably emitted by means of a laser device. In a further embodiment the fluid of the volume increment can contain one or more starting compounds, typically monomers, of one or more polymers, and after application of the voxel a polymerization is initiated by suitable means such as, again, light, heat or other polymerization initiators curing the applied voxel and binding it or adhering it, respectively, to adjacent voxels.

Suitable carrier materials being flowable at the printing temperature in which the pharmaceutical active agent(s) are present are e.g., carriers generally usable in hot melt extrusion (HME) such as low-melting waxes and polymers. The HME mixture, or in general the mixture of the volume increment, can contain, besides the low-melting carrier, further process agents and aids such as binders, emollients, antioxidants, perfumes, sweeteners or similar. Suitable HME carriers and emollients are disclosed e.g., in Crowley et al. (2007) Drug Development and Industrial Pharmacy, 33,909-926 (carriers: pages 917 to 919, in particular Table 1; emollients: pages 917 and 920, in particular Table 2) wherein the present description expressis verbis includes the mentioned passages by reference.

The method according to the invention is not limited to the complete de novo assembly of dosage forms or medical devices. The method can also be applied to objects already provided on the building device. Included are e.g., dosage forms produced conventionally or in other manner, which are e.g., to be modified by the method of the invention or active agent-free objects (also denoted as placebo carriers) on which active agent-containing volume increments are printed according to the mode of the invention. For example, active agent-free films or other flat materials such as edible wafer paper can be provided for producing ODF (“orally degradable film” or “orally dissolvable film”) products. In a further embodiment, volume increments containing e.g., active agents promoting wound healing can printed on provided plaster materials. In further embodiments API-containing volume increments can be printed on placebo carriers produced e.g., by fused layer modeling, followed by printing of solvent-containing liquids whereby the printed layers can alternate.

The present additive 3D printing method is preferably carried computerized. Thus, in step (i) a calculated three-dimensional picture of the object to be printed is typically generated by means of a well-established CAD program. The computer-generated representation of the object to be printed can also be produced by scanning of a pre-existing dosage form. During the present method, the computer-generated model picture is then subdivided into the desired, generally freely selectable volume increments (voxels) wherein the resolution of the real object is higher the smaller the volume increments are. For example, active agent, carrier or base substance and carrier or base compositions, respectively, and/or further aids such as color substances and optionally further required materials as well as their amounts (concentration in the volume increment) can be assigned to each individual volume increment and ultimately printed. Suitable printing devices for the present method are described e.g., in US 2017/0368755 A1 and U.S. Pat. No. 6,070,107.

In a particularly preferred embodiment, the method according to the invention further comprises the application of at least one colored substance by use of 2D and/or 3D printing, preferably also by printing of corresponding voxels, preferably having a small volume, according to the present method, and/or by a different method e.g., by two-dimensional printing such as according to ink printing, on the object or on at least a region of the object in a manner such that the applied substance forms at least one information structure visible on the object. The colored substance(s) can be applied separately from the active agent-containing volume increment(s). It is preferred that the color substance(s) are applied together with the volume increments containing pharmaceutical active agents. In one embodiment a respective substance can label those parts or regions on which the respective active agent has been applied. This embodiment can thus transport the information on the active agents present in the complete object and their distribution by color coding. In a further elaboration of this embodiment of the invention, different amounts or concentrations, respectively, of the respective active agent can be deposited in active agent-containing regions which is reflected by the concentration of the respective color substance. Different color substances can, of course, be mixed in e.g., a voxel according to the invention such that the complete visible spectrum can be employed by corresponding selection of the mixture(s).

According to the invention, the term “color substance” also includes luminescent, particularly fluorescent, substances.

The information structure generated by the color substance(s) can represent various information wherein several different information structures through different color substances can be employed, which can be selected and combined in an essentially free fashion. In particular, it is envisaged according to the invention that the at least one information structure encodes information on the type of or the active agents printed in the object and/or on the amount(s) of active agent(s) present in the object and/or on the intended time point or time region of taking of the dosage form and/or on the intended date of taking of the dosage form and/or on patient-related data (such as name, age, sex, medication and disease(s) of the patient) and/or on the cost center and/or on the attending physician and/or on the pharmaceutical company providing the dosage form and/or on the medical unit dispensing the object such as a dosage form or a medical device.

The information structure can be selected from a wide variety of applications. For example, the substance(s) can be printed in the form of QR codes, characters and/or numbers. Of course, various patterns such as lines, grids, points, two-dimensional patterns etc. can be printed whereby the preferred embodiment of voxel printing provides generally the most diverse possibilities. The printed image of the code can therefore contain the active agent and at the same time encode, as described above, the desired data on patient, physician, pharmacist and/or qualified medical or pharmaceutical professionals.

It is evident to the skilled person that depending on the selected specific production process the optionally present color substance(s) can be present together with the pharmaceutical active agent(s) in the respective printed base composition. In the present method it is preferred that the color substance (or more thereof) is present together with the active agent(s) in the building substance which is intended for a given volume increment.

As outlined above, the dosage form can contain various information structures, preferably such information structures mentioned for the above-described method.

Active agent-containing objects printable by means of the method are, in particular, semi-solid or solid pharmaceutical dosage forms such as tablets, capsules, implants, plasters, suppositories or thin films. Tablets producible by means of the method according to the invention are manifold and include oblong shape tablets, lozenges, implantable tablets, multiapplication tablets, disperse tablets, retard tablets, vaginal tablets and suppositories, eye tablets, coated tablets, matrix tablets, chewable tablets, film-coated tablets, modified-release tablets, lacquer tablets and enteric coated and drug abuse-deterrent tablets.

Further particularly suitable objects are medical devices such as active agent-containing topical drug forms, contact lenses, plasters, which preferably release the active agent(s) for topical application.

The present invention is also directed to the two- or three-dimensional objects (preferably those mentioned above) produced by the method, wherein the objects contain at least one pharmaceutical active agent.

### BRIEF DESCRIPTION OF THE DRAWINGS

The present invention is further illustrated by the enclosed Figures:

FIG. 1 shows a schematic representation of a cross section of a three-dimensional object subdivided (hereinafter also denoted as “voxelized”) into individual volume increments (quadratic in cross section).

FIG. 2 shows a schematic representation of a three-dimensional object subdivided (thus, voxelized) into cubic volume increments wherein the resolution is lower (thus, coarser resolution) due to larger voxels in FIG. 2A, and the resolution in FIG. 2B is higher (thus, finer resolution) due to subdivision into smaller volume increments.

FIG. 3 shows a schematic representation of a cross section of a three-dimensional object in which volume increments (represented in the cross section as squares) without active agent and active agent-containing voxels (shown as lighter voxels) alternate with volume increments with active agent (shown as darker voxels) resulting in a concentration of voxels with active agent of e.g., 50%.

FIG. 4 shows a schematic representation of a cross section of a three-dimensional object in which individual voxels contain active agent (shown dark) such that 25% of the voxels contain active agent. The ratio of active agent-containing voxels to active agent-free voxels or the volume percentage of the voxels containing the different active agents, respectively, can be selected arbitrarily between 0.1 and 99.9%.

FIG. 5 shows a schematic representation of a cross section of a three-dimensional object in which active agent-containing voxels (dark) are concentrated in the inside and surrounded by active agent-free voxels (light) such that they can protect the active agent-containing voxels from the environment such as a body fluid. The release of the active agent can be regulated by such a design. In other embodiments the release regulation can be attained, as disclosed above, by means of pH dependently soluble or degradable, respectively, polymers. By the presently disclosed and described practice the different concentrations and materials can result in dosage forms having high resolution. Thus, the dark voxels of FIG. 5 may e.g., correspond to FIG. 3, and the light voxels may correspond to FIG. 4. In this manner, it is also possible to produce e.g., tablets having abuse-deterrent characteristics as already described above.

FIG. 6 shows a schematic representation of an object producible according to the invention which further elaborates the embodiment illustrated in FIG. 5. Voxels having a first active agent (blue) are surrounded by voxels having a second active agent (red) which in turn are surrounded by active agent-free voxels. This embodiment can also be designed by not using different active agents but different concentrations of active agent.

FIG. 7 shows a schematic representation of a cross section of a three-dimensional object in which a concentration gradient of the active agent is built in the object by decreasing the density of the active agent-containing voxels from left to right.

FIG. 8 shows a schematic representation of an object producible according to the invention in which voxels having three different active agents or concentrations of active agent (blue, red, green) are dispersed in the object and surrounded by active agent-free voxels.

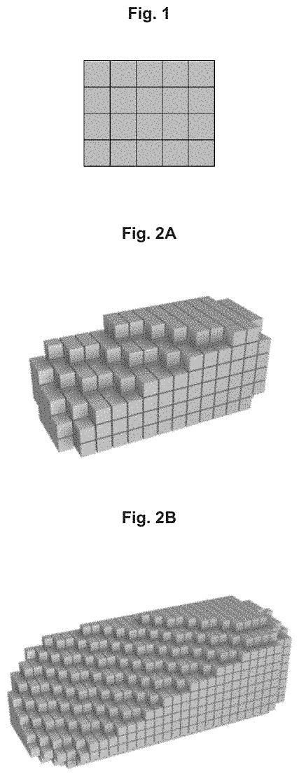

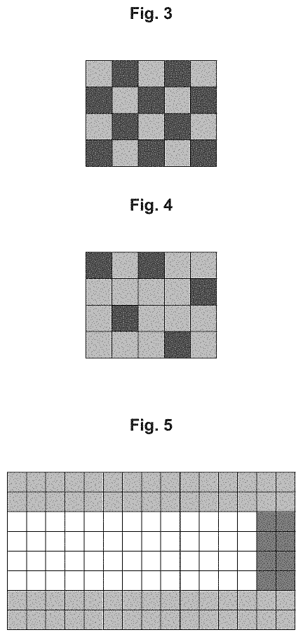

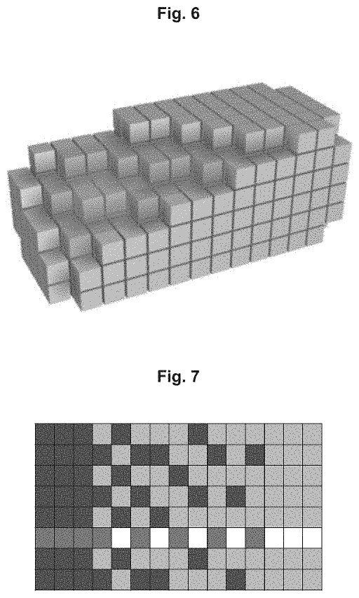

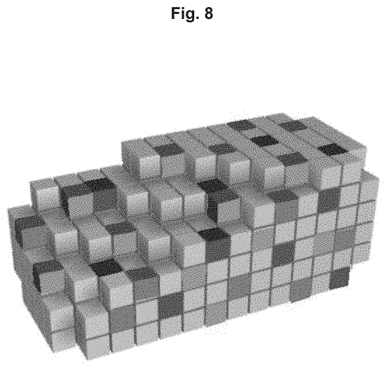

### DETAILED DESCRIPTION OF THE INVENTION

According to exemplary embodiments, the method of the invention can provide e.g., an adjustment of the release of active agent according to the physiological requirements in patients having heart conditions or hypertension. For example, a cholesterol-lowering agent (such as a statin) and two antihypertensives (e.g., Sartan and a beta-blocker) can be printed together in one dosage from in such a manner that the release of the active agents optimally corresponds to the physiological conditions of the patient. In case of an administration of the dosage form in the morning the antihypertensives can be released quickly whereas the release of the cholesterol-lowering agent is retarded for 12 to 16 hours in order to reduce the cholesterol synthesis during the night.

In further examples of the method according to the invention a titration of patients having newly diagnosed diseases such as Parkinson's disease, multiple sclerosis, heart attack, stroke or transplanted patients and applications in geriatrics and pediatrics, respectively, can be provided. In all of these cases the patient specific dosage and release of active agents optimize the effectiveness and reduce side effects.

The present invention relates particularly to the following aspects and preferred embodiments:
1. A method for the production of an object containing at least one pharmaceutical active agent, the method comprising:
(i) generating a two- or three-dimensional representation of the object to be produced using predefined volume increments;
(ii) printing a predefined volume increment on a building platform or on an object provided on the building platform;
(iii) printing a further volume increment in a manner such that volume increments at least partially contact or overlap each other; and
(iv) repeating steps (ii) and (iii) until the object is generated;
wherein at least one of the volume increments contains at least one pharmaceutical active agent and the volume increments comprise a base composition or base substance being flowable at a printing temperature acceptable for the at least one pharmaceutical agent, which base composition or base substance solidifies after printing of the respective volume increment and/or are glued.
2—The method of point 1. wherein the steps (ii) and (iii) are carried out in a manner such that the volume increments are printed in layers.
3. The method of point 1. or 2. wherein each volume increment contains an active agent.
4. The method of point 1. or 2. wherein different volume increments comprise different active agents and/or different amounts of active agent and/or different base compositions or base substances, respectively.
5. The method according to any one of the preceding points wherein the shape of the volume increments is the same or different.
6. The method according to any one of the preceding points wherein the volume of the volume increments is the same of different.
7. The method of point 6 wherein the volume of the volume increments is different in a manner such that the volume of the volume increments increases from the outside to the inside of the printed object.
8. The method according to any one of the preceding points wherein the shape of the volume increments is selected from the group consisting of drops, spheres, points, cylinders, cubes, and cuboids.
9. The method according to any one of the preceding points wherein active agent-containing volume increments are configured and printed in a manner such that at least a first group of contacting or overlapping active agent-containing volume increments contain the same amount of active agent, and at least a second group of contacting volume increments contain an amount of active agent being different from the amount of active agent of the first group.
10. The method according to any one of the preceding points wherein at least one group of active agent-containing volume increments is present containing a first pharmaceutical active agent and at least a second group of active agent-containing volume increments is present containing a second active agent which is different from the first active agent.
11. The method of point 10. wherein the volume increments of the first and/or the second group are printed in a manner such that the volume increments of the respective group contact each other.
12. The method according to any one of the preceding points wherein the volume increments are printed in a manner such that active agent-containing volume increments in the object form one or more groups surrounded by active agent-free volume increments which shield the active agent-containing volume increments from the external environment.
13. The method of point 12. wherein the active agent-containing volume increments in the object form an active agent-containing core around which active agent-free volume increments are arranged.
14. The method of point 12. wherein the volume increments are printed in a manner such that active agent-containing volume increments form several groups which are respectively surrounded by active agent-free volume increments.
15. The method of point 13. or 14. wherein the active agent-free volume increments contain a substance at least limiting the effect of the active agent(s) of the active agent-containing volume increments when the printed object is disintegrated.
16. The method according to any one of points 13. to 15. wherein the active agent(s) has/have addiction potential.
17. The method of point 13. wherein the active agent-free volume increments are printed in a manner such that they form one or more layers around an active agent-containing core, which layer(s) is/are degraded pH dependently in the intestine of a patient,
18. The method according to any one of points 14. to 17. wherein active-agent free volume increments effect a retarded release of the active agent(s).

19. The method according to any one of points 13. to 18. wherein active agent-free volume increments provide the printed object with gastric juice resistance.

20. The method according to any one of the preceding points wherein the active agent-containing object is a semi-solid or solid pharmaceutical dosage form.

21. The method of point 20. wherein the dosage form is selected from the group consisting of tablets, capsules, suppositories, plasters or thin films.

22. The method of point 21. wherein the tablets are selected from the group consisting of oblong shape tablets, lozenges, implantable tablets, multiapplication tablets, disperse tablets, retard tablets, lacquer tablets and enteric coated tablets, vaginal tablets and suppositories, eye tablets, coated tablets, matrix tablets, chewable tablets, film-coated tablets, modified-release tablets.

23. The method according to any one of points 1. to 16. wherein the object is a medical device.

24. Three-dimensional object containing at least one pharmaceutical active agent, the object being produced by the method according to any one of the preceding points.

1. A method for the production of an object containing at least one pharmaceutical active agent, the method comprising:
(i) generating a two- or three-dimensional representation of the object to be produced using predefined volume increments;
(ii) printing a predefined volume increment on a building platform or on an object provided on the building platform;
(iii) printing a further volume increment in a manner such that volume increments at least partially contact or overlap each other; and
(iv) repeating steps (ii) and (iii) until the object is generated;
wherein at least one of the volume increments contains at least one pharmaceutical active agent and the volume increments comprise a base composition or base substance being flowable at a printing temperature acceptable for the at least one pharmaceutical agent, which base composition or base substance solidifies after printing of the respective volume increment and/or are glued.

2. The method of claim 1 wherein the steps (ii) and (iii) are carried out in a manner such that the volume increments are printed in layers.

3. The method of claim 1 wherein each volume increment contains an active agent.

4. The method of claim 1 wherein different volume increments comprise different active agents and/or different amounts of active agent and/or different base compositions or base substances, respectively.

5. The method according claim 1, wherein the shape of the volume increments is the same or different.

6. The method according to claim 1, wherein the volume of the volume increments is the same or different.

7. The method of claim 6 wherein the volume of the volume increments is different in a manner such that the volume of the volume increments increases from the outside to the inside of the printed object.

8. The method according to claim 1, wherein the shape of the volume increments is selected from the group consisting of drops, spheres, points, cylinders, cubes, and cuboids.

9. The method according to claim 1, wherein active agent-containing volume increments are configured and printed in a manner such that at least a first group of contacting or overlapping active agent-containing volume increments contain the same amount of active agent, and at least a second group of contacting volume increments contain an amount of active agent being different from the amount of active agent of the first group.

10. The method according to claim 1, wherein at least one group of active agent-containing volume increments is present containing a first pharmaceutical active agent and at least a second group of active agent-containing volume increments is present containing a second active agent which is different from the first active agent.

11. The method of claim 10 wherein the volume increments of the first and/or the second group are printed in a manner such that the volume increments of the respective group contact each other.

12. The method according to claim 1, wherein the volume increments are printed in a manner such that active agent-containing volume increments in the object form one or more groups surrounded by active agent-free volume increments which shield the active agent-containing volume increments from the external environment.

13. The method of claim 12 wherein the active agent-containing volume increments in the object form an active agent-containing core around which active agent-free volume increments are arranged.

14. The method of claim 12 wherein the volume increments are printed in a manner such that active agent-containing volume increments form several groups which are respectively surrounded by active agent-free volume increments.

15. The method of claim 13, wherein the active agent-free volume increments contain a substance at least limiting the effect of the active agent(s) of the active agent-containing volume increments when the printed object is disintegrated.

16. The method according to claim 13, wherein the active agent(s) has/have addiction potential.

17. The method of claim 13 wherein the active agent-free volume increments are printed in a manner such that they form one or more layers around an active agent-containing core, which layer(s) is/are degraded pH dependently in the intestine of a patient.

18. The method according to claim 14, wherein the active-agent free volume increments effect a retarded release of the active agent(s).

19. The method according to claim 13, wherein the active agent-free volume increments provide the printed object with gastric juice resistance.

20. The method according to claim 1, wherein the active agent-containing object is a semi-solid or solid pharmaceutical dosage form.

21. The method of claim 20 wherein the dosage form is selected from the group consisting of tablets, capsules, suppositories, plasters or thin films.

22. The method of claim 21 wherein the tablets are selected from the group consisting of oblong shape tablets, lozenges, implantable tablets, multiapplication tablets, disperse tablets, retard tablets, lacquer tablets and enteric coated tablets, vaginal tablets and suppositories, eye tablets, coated tablets, matrix tablets, chewable tablets, film-coated tablets, modified-release tablets.

23. The method according to claim 1, wherein the object is a medical device.

24. A three-dimensional object containing at least one pharmaceutical active agent, the object being produced by the method according to claim 1.
